(19) United States
(12) Patent Application Publication
(10) Pub. No.: US 2018/0289287 A1
(43) Pub. Date: Oct. 11, 2018

(54) TREATMENT APPARATUS AND METHOD FOR TREATING A GAIT IRREGULARITY OF A PERSON

(71) Applicant: KONINKLIJKE PHILIPS N.V., EINDHOVEN (NL)

(72) Inventors: CHARLES FREDERIK SIO, EINDHOVEN (NL); MARC ANDRE DE SAMBER, LOMMEL (BE); RONALDUS MARIA AARTS, GELDROP (NL)

(73) Assignee: KONINKLIJKE PHILIPS N.V., Eindhoven (NL)

(21) Appl. No.: 15/766,070

(22) PCT Filed: Sep. 28, 2016
(86) PCT No.: PCT/EP2016/073026

§ 371 Date: Apr. 5, 2018

(30) Foreign Application Priority Data
Oct. 8, 2015 (EP) 15188927.6

(51) Int. Cl. A61B 5/11; A61B 5/00

(52) U.S. Cl. CPC A61B 5/112; A61B 5/486; G16H 50/20; A61H 2201/165; A61B 5/4848

(57) ABSTRACT

The invention relates to a treatment apparatus for treating a gait irregularity of a person (1). The treatment apparatus (10) comprises a gait irregularity monitoring unit (11) for detecting a gait irregularity of the person, (a) cueing unit(s) (15, 16, 17), and a cueing control unit (14) for controlling the cueing unit(s) to provide, in response to the detection of the gait irregularity, a first cue set for treating the gait irregularity to the person. The gait irregularity monitoring unit monitors the effect of the providing of the first cue set to the person and determines whether the detected gait irregularity is being overcome. The cueing control unit controls the cueing unit(s) to provide a different second cue set to the person, if it has been determined that the detected gait irregularity has not been overcome. This improves the likelihood of being able to effectively treat the detected gait irregularity.

### FIELD OF THE INVENTION

The invention relates to a treatment apparatus for treating a gait irregularity of a person. The invention relates further to a corresponding treatment method for treating a gait irregularity of a person and to a computer program for controlling the treatment apparatus.

### BACKGROUND OF THE INVENTION

WO 2010/124013 A1 discloses a device and a method for treating persons with Freezing of Gait (FoG) disorders. FoG is a major mobility problem where walking becomes hesitant and may cease entirely. This is described by patients as if their feet are ‘glued to the floor’ and occurs in a large portion of the Parkinson's Disease (PD) patient population. Episodes are transient, but freezing may be awkward or dangerous in several circumstances, increases the risk of falls and ultimately enhances the chance of nursing home placement. An episode of FoG may be overcome by providing an external pattern, often in the form of auditory or visual cues which encourage the feet to step up and over, as if unsticking from glue, rather than stepping forward, as with regular walking. To this end, the known device comprises a motion sensor for sensing the movement of a person, wherein the device automatically provides an auditory cue when a change in regularity of gait or akinesia is detected.

However, there exist significant intra- and inter-individual differences in the treatment effect achievable by a certain cue. Moreover, the treatment effect of a cue may wear off over time due to habituation processes and a progression of the disease may render previously successful cues unsuccessful over time. Thus, the known device may not always be effective in treating an episode of FoG.

US 2009/0264789 A1 discloses that a set of therapy parameter values is selected based on a patient state, where the patient state comprises a speech state or a mixed patient state including the speech state and at least one of a movement state or a sleep state. In this way, therapy delivery is tailored to the patient state, which may include one or more patient symptoms specific to the patient state.

US 2009/0099627 A1 discloses that a movement state of a patient is detected based on brain signals, such as an electroencephalogram (EEG) signal. In some examples, a brain signal within a dorsal-lateral prefrontal cortex of a brain of the patient indicative of prospective movement of the patient may be sensed in order to detect the movement state. The movement state may include the brain state that indicates the patient is intending on initiating movement, attempting to initiate movement or is actually moving.

US 2009/0082829 A1 discloses that a patient controls the delivery of therapy through volitional inputs that are detected by a bio signal within the brain. The volitional patient input may be directed towards performing a specific physical or mental activity, such as moving a muscle or performing a mathematical calculation.

### SUMMARY OF THE INVENTION

It is an object of the invention to provide a treatment apparatus for treating a gait irregularity of a person, which can have an improved effectiveness in treating the gait irregularity. It is a further object of the invention to provide a corresponding treatment method for treating a gait irregularity of a person and a computer program for controlling the treatment apparatus.

In a first aspect of the invention, a treatment apparatus for treating a gait irregularity of a person is presented, wherein the gait irregularity is Freezing of Gait (FoG), wherein the treatment apparatus comprises:
a gait irregularity monitoring unit for detecting a gait irregularity of the person,
one or more cueing units, and
a cueing control unit for controlling, in response to the detection of the gait irregularity, the one or more cueing units to provide a first cue set of one or more cues for treating the detected gait irregularity to the person,
wherein the gait irregularity monitoring unit is adapted to monitor the effect of the providing of the first cue set to the person and to determine whether the detected gait irregularity is being overcome, and
wherein the cueing control unit is adapted to control the one or more cueing units to provide a different second cue set of one or more cues to the person, if it has been determined that the detected gait irregularity has not been overcome.

Since the gait irregularity monitoring unit monitors the effect of the providing of the first cue set to the person and determines whether the detected gait irregularity is being overcome, and since the cueing control unit controls the one or more cueing units to provide a different second cue set to the person, if it has been determined that the detected gait irregularity has not been overcome, it is possible to try different cue sets on the person. This improves the likelihood of being able to effectively treat the detected gait irregularity when intra- and inter-individual differences in the treatment effect achievable by a certain cue exist, when the treatment effect of a cue may wear off over time due to habituation processes, and when a progression of the disease may render previously successful cues unsuccessful over time. Moreover, the duration until the detected gait irregularity is overcome may be shorter.

To give an example, persons with FoG disorders are a heterogeneous population with regards to how often they experience FoG, the severity, the factors that induce FoG, and the tricks or cues that can help them to overcome an episode of FoG. For instance, S. Rahman et al., “The Factors that Induce or Overcome Freezing of Gait in Parkinson's Disease”, Behavioural Neurology, Vol. 19, No. 3, pages 127 to 136 (2008) report results from a postal survey of 130 PD patients of which 72% reported FoG. The results show that FoG most commonly lasts a couple of seconds, but episodes can occasionally exceed 30 seconds. As FoG-inducing factors, a large variety of situations or conditions, such as turning around, fatigue, being in a confined space, being in a stressful situation, walking in a narrow space, being in a crowd, going through doorways, et cetera, are mentioned. For overcoming a FoG episode, tricks like paying attention to every step, taking longer steps, altering the distribution of body weight, et cetera have been reported as being helpful. It is also known that with FoG, the effectiveness of tricks/cues may vary from episode to episode for an individual and the treatment effect of a trick/cue may wear off over time. Moreover, the wrong cue, or the right cue at the wrong time, or too many cues at the same time, may even induce episodes of FoG. Against this background, the possibility to try different cue sets on the person makes it more likely that the ‘right’ cue set is found for a specific FoG episode.

In the context of this specification, the terms “cue” and “cueing” are understood as referring to the application of temporal and/or spatial external stimuli associated with the initiation and/or ongoing facilitation of gait motor activity.

It is preferred that the first and the second cue set differ (i) in a modality of at least one cue, and/or (ii) in that the second cue set comprises at least one cue of an additional modality compared to the first cue set or vice versa, and/or (iii) in that an information provided by at least one cue of a given modality differs between the first and the second cue set.

In the context of this specification, the term “modality” is understood as referring to the mode of delivery of a given cue together with the specific sense the cue is addressing. For instance, an auditory cue is generally delivered by an acoustic transducer (e.g., a loudspeaker, a headphone, et cetera) and addresses the sense of hearing, whereas a visual cue is generally delivered by an optical transducer (e.g., a laser pointer, an image or video projector, et cetera) and addresses the sense of vision. These two cues are considered to be of a different modality. However, also a visual cue delivered by a laser pointer is considered to be of a different modality than a visual cue delivered by an image or video projector, even though they both address the sense of vision. Likewise, an electrical stimulation cue delivered, for instance, by an electrode patch is considered to be of a different modality than a tactile stimulation cue delivered, for instance, by a vibration device, even though they both address the somatic senses of the skin. The term “information”, on the other hand, is understood in the context of the present specification as referring to the characteristics of the stimulus provided by a cue of a given modality. To give an example, a first auditory cue delivered by a headphone could be a rhythmic sound, such as drumming, a second auditory cue delivered by the headphone could be a verbal command, like “Walk 1, 2, 3, 4”, and a third auditory cue delivered by the headphone could be a music sample, such as “I'm Walking” from Fats Domino. These three cues are considered to be of the same modality, but each provides a different information.

An auditory cue of a given modality may provide a given information alternately to each ear of the person. For instance, a captured voice may alternately say the command “Left”, which is delivered to the left ear of the person, and the command “Right”, which is delivered to the right ear of the person. To alternately target the left and right ear of the person can potentially provide a strong stimulus for overcoming the detected gait irregularity. A similar effect can also be achieved with visual cues. For instance, an image or video projector can be used to alternately project images of a left foot and a right foot in front of the person's feet, wherein the left foot is projected more to the left than the right foot. Of course, such alternating auditory and visual cues can also be combined to advantage in a given cue set.

It is further preferred that the cueing control unit is adapted to control the one or more cueing units to provide the first cue set to the person for a first predetermined period of time and the gait irregularity monitoring unit is adapted to determine that the detected gait irregularity has not been overcome if the detected gait irregularity has not ceased until the end of the first predetermined period of time. Preferably, the first predetermined period of time is in the range of 1 to 5 seconds, for example, 2 seconds, such that when the detected gait irregularity is still found to be present after the 2 seconds, the gait irregularity monitoring unit determines that the detected gait irregularity has not been overcome, whereupon the cueing control unit controls the one or more cueing units to provide the different second cue set to the person.

It is preferred that the first and the second cue set form part of a cue set sequence of different cue sets, wherein the cueing control unit is adapted to control the one or more cueing units to successively provide the different cue sets to the person, as long as it is determined by the gait irregularity determining unit that the detected gait irregularity has not been overcome. By doing so, the different cue sets can be tried on the person one by one until the ‘right’ cue set that is able to end the detected gait irregularity is found. On the other hand, if the gait irregularity determining unit determines at some point, for instance, after a certain number of the different cue sets have been provided to the person, that the detected gait irregularity is not present anymore, the detected gait irregularity may be considered as being overcome and the cueing may be stopped. In this case, it is also possible that the cueing control unit controls the one or more cueing units to provide the current ‘right’ cue set to the person for an additional predetermined period of time, e.g., for another 3 seconds, in order to increase the likelihood that the treatment effect is persistent.

Preferably, the treatment apparatus further comprises:
a gait irregularity qualifying unit for qualifying the detected gait irregularity based on information that is indicative of a current context of the person, and/or of past gait irregularities of the person, and/or of gait irregularities of a population of persons,
wherein the cueing control unit is adapted to construct the cue set sequence based on the outcome of the qualifying.

By doing so, it can be possible to tailor the cue set sequence to the specifics of the detected gait irregularity. For instance, the qualification of the detected gait irregularity based on information that is indicative of a current context of the person may include the qualification according to severity, the moment of onset (e.g., time of the day), physiological state of the person (e.g., heart rate), the presence of additional triggers, et cetera. Moreover, information that is indicative of past gait irregularities of the person may be used to qualify the detected gait irregularity with respect to occurrence over time, the evolution of events and their severity, et cetera. In addition, information that is indicative of gait irregularities of a population of persons can be helpful to assess the detected gait irregularity in comparison to events experienced by other persons with a comparable disease or with disease symptoms that are of a comparable type and severity. Based on the available information, the cue set sequence may then be constructed in a way that is expected to be best suited for the detected gait irregularity. For example, the order of the cue sets and/or their composition may be specifically chosen in terms of modalities and/or information for the detected gait irregularity. This can be realized, for instance, by means of (an) algorithm(s) that is/are able to analyze, e.g., by means of statistical methods, the available information and that construct(s) the cue set sequence based on the outcome of the analysis. In order to store the available information, in particular, the information that is indicative of past gait irregularities of the person, the treatment apparatus can further comprise a suitable storage unit. Also, an information communication unit can be foreseen, for instance, in order to allow the information that is indicative of gait irregularities of a population of persons to be communicated to the treatment apparatus. Of course, it can also be possible that data is partially or completely stored and/or analyzed at a remote location, e.g., a server of a manufacturer of the treatment apparatus or of a special service provider. In this case, the treatment apparatus also comprises a suitable communication interface for allowing the data to be communicated to the remote location.

The information that is indicative of a current context of the person, and/or of past gait irregularities of the person, and/or of gait irregularities of a population of persons may further be used to adjust (a) parameter(s) of at least one cue in a given cue set. The term “parameter” is understood in the context of this specification as referring to, for instance, the strength or shape of a visual cue, the loudness, frequency or pitch of an auditory cue being a rhythmic sound, the duration and frequency of a vibration stimulus, et cetera. So, for instance, it can be foreseen that the strength of a visual cue may be adjusted in accordance with a brightness level of the person's environment or that the loudness of an auditory cue may be adjusted in accordance with a loudness level of the person's environment.

It is also preferred that the treatment apparatus further comprises:
one or more context information providing units for providing the information that is indicative of the current context of the person.

The one or more context information providing units may comprise, for instance, sensing units that are adapted to sense situational aspects of the person (e.g., detection of narrow passages, turning of the person, crowded areas, et cetera), local aspects (e.g., locations where the person repeatedly experienced a gait irregularity), cognitive-effective aspects (e.g., stress, cognitive load, et cetera), physiological aspects (e.g., heart rate), and so on. On the other hand, they may also comprise units that just give, for instance, the time of the day. Additionally or alternatively, the one or more context information providing units may be adapted to obtain the information that is indicative of the current context of the person from other data sources on a higher level. For instance, data from a city-wide sensor networks that collects information on traffic situations (traffic congestion, leading to crowding disturbances for the person) may be used in combination with data from (a) sensing unit(s) that is/are adapted to sense the location of the person (e.g., a GPS unit) in order to identify contexts that might have an effect on the person's risk of experiencing a gait irregularity.

It is preferred that the one or more context information providing units comprise:
an input unit for inputting information about a medication administration of the person,
wherein the gait irregularity qualifying unit is adapted to qualify the detected gait irregularity based on information that is indicative of a current context of the person, wherein the current context of the person comprises the current medication status of the person.

This makes it possible to take the current medication status of the person into consideration in the providing of the different cue sets to the person. For instance, a visual cue may be provided to the person with a specific strength or an auditory cue being a rhythmic sound may be provided with a specific frequency that are well suited for the current medication status of the person.

It is preferred that the gait irregularity determining unit is adapted to detect and/or monitor the gait irregularity by means of an algorithm, and/or that the cueing control unit is adapted to construct the cue set sequence by means of an algorithm,
wherein the treatment apparatus further comprises:
an update unit for allowing the algorithm(s) and/or data that is input to the algorithm(s) to be updated.

This makes it possible to upgrade and improve the treatment apparatus, for instance, when more (a) efficient and reliable algorithm(s) for detecting and/or monitoring the gait irregularity become available or when new knowledge about the suitability of certain cues becomes available. The data can be, for instance, information that is indicative of gait irregularities of a population of persons. The update unit can comprise, for instance, an interface for inserting a data storage device, such as a USB (Universal Serial Bus) stick, that can be used for uploading (a) new algorithm(s) and/or data to the treatment apparatus, or it can comprise a suitable communication interface for allowing an upload from a remote location.

It is also preferred that the treatment apparatus further comprises:
an alarm communication unit for automatically communicating an alarm to a remote location, if it is determined by the gait irregularity determining unit that, after the providing of a predetermined number of the different cue sets to the person or after a second predetermined period of time, the detected gait irregularity has still not been overcome.

Stronger gait irregularities that cannot be quickly overcome can cause severe fatigue, so that especially if the person is elderly, he/she may need help in order to be able to return from a walk or the like. The remote location may be, for instance, at a person from his/her family, at a caregiver, or at a service like Lifeline. The alarm communication unit is preferably a mobile communication unit that is adapted to automatically ‘call’ a responsible person using suitable mobile communication technologies. The treatment apparatus may comprise, for instance, a hands-free speaking system, such that the person can tell the receiver of the call where he/she is currently located and how his/her current situation is. Moreover, it is possible that the treatment apparatus comprises a location determining unit, such as a GPS unit or the like, for determining the location of the person, and that the mobile communication unit is further adapted to communicate the location of the person to the remote location. The predetermined number of the different cue sets can be, for instance, 10. Similarly, the second predetermined period of time can be, for instance, 20 seconds.

It is preferred that the treatment apparatus further comprises:
a user interface for allowing the person to input a stop command and/or a sensitivity change command,
wherein the cueing control unit is adapted to stop, in response to the input of the stop command, the one or more cueing units to provide the different cue sets to the person, and/or
wherein the gait irregularity monitoring unit is adapted to change, in response to the input of the sensitivity change command, a sensitivity of the detecting of the gait irregularity of the person.

This provides the person with an ability to interfere with and/or control the functioning of the treatment apparatus, if desired or needed. For instance, if the gait irregularity monitoring unit erroneously detects a gait irregularity of the person and the cueing control unit begins with the providing of the different cue sets to the person, the person can stop this. Likewise, if the gait irregularity monitoring unit erroneously does not determine that the detected gait irregularity has been overcome, the person can also stop the providing of the different cue sets to the person. Also, if the person wants to decrease or increase the threshold for the providing of the different cue sets to the person, he/she can do so by inputting an appropriate sensitivity change command. For instance, the person may want to decrease the threshold in situations where gait irregularities are particularly inconvenient or dangerous, such as when crossing a street. On the other hand, he/she may want to increase the threshold in situations where the providing of the different cue sets to the person would be particularly inconvenient, such as when attending a lecture or a theatrical performance. In addition, the user interface may also be adapted to allow the person to turn off the treatment apparatus completely and to switch it on back again. The user interface unit can comprise one or more simple manual controls, like buttons, jog dials, or the like, it can comprise a touch interface as provided by many of today's mobile communication devices, it can comprise means for allowing a control via verbal commands, or it can comprise any other suitable kinds of controls.

It is preferred that the treatment apparatus further comprises:
one or more gait information sensing units for sensing information that is indicative of a current gait of the person,
wherein the gait irregularity monitoring unit is adapted to detect the gait irregularity based on the sensed information.

The one or more gait information sensing units are preferably wearable, such that they can be, for instance, attached to the legs of the person in order to sense the information that is indicative of the current gait of the person.

Preferably, the one or more gait information sensing units comprise: (i) an accelerometer, and/or (ii) a gyroscope, and/or (iii) a muscle tension sensor. Such gait information sensing units are particularly suited for providing the information that is indicative of a current gait of the person. For instance, a first accelerometer can be foreseen to be attached to the shank (e.g., just above the ankle) of the person and a second accelerometer can be foreseen to be attached to the thigh (e.g., just above the knee) of the person and the gait irregularity monitoring unit can be adapted to detect the gait irregularity based on the sensed acceleration information from the two accelerometers. In this example, it would also be possible, for instance, that an additional muscle tension sensor is used to further improve the sensitivity and/or the specificity of the detecting of the gait irregularity of the person by providing additional muscle tension information that can be employed by the gait irregularity monitoring unit. The additional muscle tension sensor can be foreseen to also be attached to the thigh of the person. It may be separate from the second accelerometer or they may be integrated into a single device or unit.

In a further aspect of the invention, a treatment method for treating a gait irregularity of a person is presented, wherein the gait irregularity is Freezing of Gait (FoG), wherein the treatment method comprises:
detecting a gait irregularity of the person, by a gait irregularity monitoring unit, and
controlling, in response to the detection of the gait irregularity, one or more cueing units to provide a first cue set of one or more cues for treating the detected gait irregularity to the person, by a cueing control unit,
wherein the effect of the providing of the first cue set to the person is monitored and it is determined whether the detected gait irregularity is being overcome, by the gait irregularity monitoring unit, and
wherein the one or more cueing units are controlled, by the cueing control unit, to provide a different second cue set of one or more cues to the person, if it has been determined that the detected gait irregularity has not been overcome.

In another aspect of the invention, a computer program for controlling a treatment apparatus for treating a gait irregularity of a person is presented, wherein the computer program comprises program code means for causing the treatment apparatus as defined in claim 1 to carry out the treatment method as defined in claim 14, when the computer program is run on the treatment apparatus.

It shall be understood that the treatment apparatus of claim 1, the treatment method of claim 14, and the computer program of claim 15 have similar and/or identical preferred embodiments, in particular, as defined in the dependent claims.

It shall be understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily a person using an embodiment of a treatment apparatus for treating a gait irregularity of the person,

FIG. 2 illustrates further details of the treatment apparatus shown in FIG. 1, and

FIG. 3 shows a flowchart exemplarily illustrating an embodiment of a treatment method for treating a gait irregularity of a person.

### DETAILED DESCRIPTION OF EMBODIMENTS

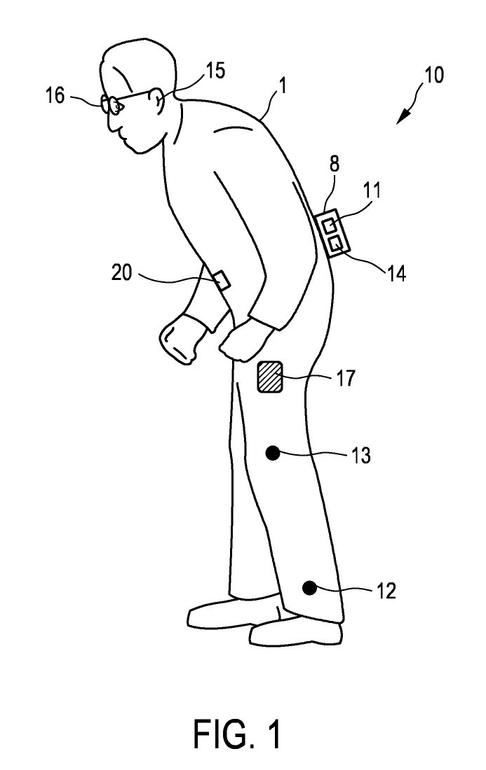

FIG. 1 shows schematically and exemplarily a person 1 using an embodiment of a treatment apparatus 10 for treating a gait irregularity of the person 1. The person 1, in this embodiment, is an elderly person having e.g. PD, wherein the gait irregularity is FoG. The treatment apparatus 10 comprises one or more, in this example, three cueing units 15, 16, and 17. A first cueing unit 15, here, an in-ear headphone, is adapted to provide an auditory cue to the person 1, a second cueing unit 16, here, a laser pointer arranged at the spectacles of the person 1, is adapted to provide a visual cue to the person 1, and the third cueing unit 17, here, an electrode patch, is adapted to provide an electrical stimulation cue to the thigh muscles of the person 1. The auditory cue can be or comprise, for instance, a rhythmic sound, such as drumming, a verbal command, like “Walk 1, 2, 3, 4” or a music sample, such as “I'm Walking” from Fats Domino, reproduced via the in-ear headphone 15. The visual cue is, in this example, a light dot projected by the laser pointer 16 in front of the person's feet. The treatment apparatus 10 further comprises a gait irregularity monitoring unit 11 for detecting the gait irregularity of the person 1 as well as one or more, in this example, two gait information sensing units 12 and 13 for sensing information that is indicative of a current gait of the person 1, wherein gait irregularity monitoring unit 11 is adapted to detect the gait irregularity based on the sensed information. The two gait information sensing units 12 and 13, here, are both accelerometers, wherein a first accelerometer 12 is attached to the shank (e.g., just above the ankle) of the person 1 and the second accelerometer 13 is attached to the thigh (e.g., just above the knee) of the person 1. For attaching the two accelerometers 12 and 13 to the person 1, elasticized straps and Velcro may be used. The gait irregularity monitoring unit 11, here, is included in a housing 8 that is worn by the person 1 at his belt. The communication between the two accelerometers 12 and 13 and the gait irregularity monitoring unit 11 can be performed, for instance, by means of a wired connection or, preferably, by using a suitable wireless communication technique.

The gait irregularity monitoring unit 11 is adapted to detect the gait irregularity based on the sensed acceleration information from the two accelerometers 12 and 13 (gait information sensing units). It may thereby use an approach as described, for example, in Bachlin et al., “Potentials of enhanced context awareness in wearable assistants for Parkinson's disease patients with the freezing of gait syndrome”, in Proceedings of 16th International Symposium on Wearable Computers, Linz, Austria, pages 123 to 130 (2009).

The treatment apparatus 10 further comprises a cueing control unit 14 for controlling, in response to the detection of the gait irregularity, the in-ear headphone 15, the laser pointer 16, and the electrode patch 17 (cueing units) to provide a first cue set of one or more cues for treating the detected gait irregularity to the person 1. Here, the cueing control unit 14 is also included in the housing 8 that is worn by the person 1 at his belt. The communication between the cueing control unit 14 and the three cueing units 15, 16, and 17 can be performed, for instance, by means of a wired connection or, preferably, by using a suitable wireless communication technique.

In this example, the first cue set consists of the verbal command “Walk 1, 2, 3, 4”, reproduced by the in-ear headphone 15, and the light dot, projected by the laser pointer 16. In contrast, the electrical stimulation cue that can be provided by the electrode patch 17 is not included in the first cue set.

The gait irregularity monitoring unit 11 is adapted to monitor the effect of the providing of the first cue set, here, the verbal command “Walk 1, 2, 3, 4” and the light dot, to the person 1 and to determine whether the detected gait irregularity is being overcome. The cueing control unit 14 is adapted to control the one or more cueing units 15, 16, and 17 to provide a different second cue set of one or more cues to the person 1, if it has been determined that the detected gait irregularity has not been overcome.

In this embodiment, the cueing control unit 14 is adapted to control the in-ear headphone 15, the laser pointer 16, and the electrode patch 17 (cueing units) to provide the first cue set to the person for a first predetermined period of time, here, 2 seconds, and the gait irregularity monitoring unit 11 is adapted to determine that the detected gait irregularity has not been overcome if the detected gait irregularity has not ceased until the end of the first predetermined period of time.

In this example, the second cue set consists of the verbal command “Walk 1, 2, 3, 4”, reproduced by the in-ear headphone 15, the light dot, projected by the laser pointer 16, and the electrical stimulation provided by the electrode patch 17. In other words, the first and the second cue set differ in that the second cue set comprises a cue of an additional modality compared to the first cue set. Additionally or alternatively, however, the first and the second cue set could also differ in a modality of at least one cue, and/or in that the first cue set comprises at least one cue of an additional modality compared to the second cue set, and/or in that an information provided by at least one cue of a given modality differs between the first and the second cue set.

The first and the second cue set, here, form part of a cue set sequence of different cue sets, wherein the cueing control unit 14 is adapted to control the in-ear headphone 15, the laser pointer 16, and the electrode patch 17 (cueing units) to successively provide the different cue sets to the person 1, as long as it is determined by the gait irregularity determining unit 11 that the detected gait irregularity has not been overcome.

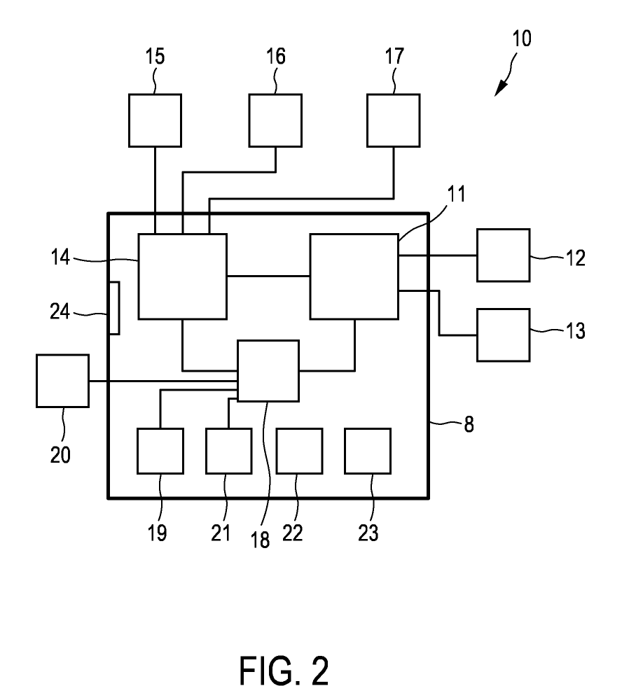

Further details of the treatment apparatus 10 shown in FIG. 1 are illustrated in FIG. 2. The treatment apparatus 10 comprises the elements that have already been explained with reference to FIG. 1 above: the gait irregularity monitoring unit 11 and the cueing control unit 14 included in the housing 8; the in-ear headphone 15, the laser pointer 16, and the electrode patch 17 (cueing units), and; the two accelerometers 12 and 13 (gait information sensing units).

In addition, the treatment apparatus 10, here, further comprises a gait irregularity qualifying unit 18 for qualifying the detected gait irregularity based on information that is indicative of a current context of the person 1. To this end, the treatment apparatus 10 further comprises one or more, in this example, primarily two context information providing units 19 and 20 for providing the information that is indicative of the current context of the person 1. Here, a first context information providing unit 19 is adapted to just give the time of the day, and the second context information providing unit 20 is a turning sensing unit that is adapted to sense a turning of the person 1. In particular, the turning sensing unit 20 is a gyroscope that is worn by the person at his belt, as also shown in FIG. 1.

In this embodiment, the cueing control unit 14 is adapted to construct the cue set sequence based on the outcome of the qualifying. This makes it possible to tailor the cue set sequence to the specifics of the detected gait irregularity. In particular, the order to the cue sets and/or their composition is specifically chosen in terms of modalities and/or information for the detected gait irregularity. Additionally or alternatively, the qualifying can also be based on information that is indicative of past gait irregularities of the person 1 and/or of gait irregularities of a population of persons. In order to store the available information, in particular, the information that is indicative of past gait irregularities of the person 1, the treatment apparatus 10 can then further comprise a suitable storage unit (not shown in the figure). Also, an information communication unit (also not shown in the figure) can be foreseen, for instance, in order to allow the information that is indicative of gait irregularities of a population of persons to be communicated to the treatment apparatus 10.

The one or more context information providing units, here, further comprise an input unit 21 for inputting information about a medication administration of the person 1, wherein the current context of the person 1 comprises the current medication status of the person 1.

In this embodiment, the gait irregularity determining unit 11 is adapted to detect and monitor the gait irregularity by means of an algorithm, and the cueing control unit 14 is adapted to construct the cue set sequence by means of an algorithm. In this case, the treatment apparatus 10 further comprises an update unit 22 for allowing the algorithms and/or data that is input to the algorithms to be updated. The data can be, for instance, information that is indicative of gait irregularities of a population of persons. The update unit, here, comprises an interface for inserting a data storage device, such as a USB (Universal Serial Bus) stick, that can be used for uploading (a) new algorithm(s) and/or data to the treatment apparatus. Additionally or alternatively, it can also comprise a suitable communication interface for allowing an upload from a remote location.

The treatment apparatus 10 further comprises, in this embodiment, an alarm communication unit 23 for automatically communicating an alarm to a remote location, if it is determined by the gait irregularity determining unit 11 that, after the providing of a predetermined number of the different cue sets to the person 1, the detected gait irregularity has still not been overcome. The alarm communication unit 23, here, is a mobile communication unit that is adapted to automatically ‘call’ a responsible person using suitable mobile communication technologies. The treatment apparatus 10 comprises a hands-free speaking system (not shown in the figure), such that the person 1 can tell the receiver of the call where he is currently located and how his current situation is. In this example, the predetermined number of the different cue sets is 10.

Here, the treatment apparatus 10 further comprises a user interface 24 for allowing the person 1 to input a stop command and a sensitivity change command. In this example, the cueing control unit 14 is adapted to stop, in response to the input of the stop command, the in-ear headphone 15, the laser pointer 16, and the electrode patch 17 (cueing units) to provide the different cue sets to the person 1, and the gait irregularity monitoring unit 11 is adapted to change, in response to the input of the sensitivity change command, a sensitivity of the detecting of the gait irregularity of the person 1. The user interface unit 24, here, comprises one or more simple manual controls, like buttons, jog dials, or the like (not shown in detail in the figure).

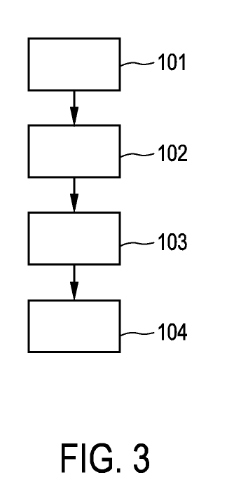

In the following, an embodiment of a treatment method for treating a gait irregularity of a person will exemplarily be described with reference to a flowchart shown in FIG. 3. The treatment method can be performed, for instance, with the treatment apparatus 10 described with reference to FIGS. 1 and 2.

In step 101, a gait irregularity of the person 1 is detected, by a gait irregularity monitoring unit 11.

In step 102, in response to the detection of the gait irregularity, one or more cueing units 15, 16, and 17 are controlled to provide a first cue set of one or more cues for treating the detected gait irregularity to the person 1, by a cueing control unit 14.

In step 103, the effect of the providing of the first cue set to the person 1 is monitored and it is determined whether the detected gait irregularity is being overcome, by the gait irregularity monitoring unit 11.

In step 104, the one or more cueing units 15, 16 and 17 are controlled, by the cueing control unit 14, to provide a different second cue set of one or more cues to the person 1, if it has been determined that the detected gait irregularity has not been overcome.

While in the embodiment of the treatment apparatus 10 described with reference to FIGS. 1 and 2 above, the treatment apparatus 10 comprises three cueing units, namely, an in-ear headphone 15, a laser pointer 16, and an electrode patch 17, other embodiments can comprise a different number of cueing units. For example, in one embodiment, a vibration unit for providing a tactile stimulation, such as a vibration, to the person's feet in the rhythm of the desired walking pace could additionally be provided in the soles of the shoes of the person. In another embodiment, video glasses for showing a different visual cue, such as a red line or a set of walking feet over the actual surroundings of the person could be provided.

Embodiments of the treatment apparatus can be realized in the form of an integrated wearable device. For instance, such a device can comprise the gait irregularity monitoring unit, the cueing control unit, an accelerometer as a gait information sensing unit, one or more cueing units, such as a loudspeaker for providing an auditory cue to the person and an electrode patch for providing an electrical stimulation cue to the muscles of the person, and, optionally, the gait irregularity qualifying unit and/or other elements, all integrated into a joint housing. The device can be adapted, for instance, to be worn by the person at his/her shank (e.g., just above the ankle).

Embodiments of the treatment apparatus can further include a functionality of providing guidance to a person experiencing gait irregularities. For instance, a suitable user interface can be provided for reminding the person to take his/her medication or to take any other preventive or reactive personal action related to episodes of the gait irregularity.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the detecting of the gait irregularity of the person, the control of the one or more cueing units to provide a cue set of one or more cues to the person, the monitoring of the effect of the providing of a cue set of one or more cues to the person, et cetera, performed by one or several units or devices can be performed by any other number of units or devices. These procedures and/or the control of the treatment apparatus in accordance with the treatment method can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The invention relates to a treatment apparatus for treating a gait irregularity of a person. The treatment apparatus comprises a gait irregularity monitoring unit for detecting a gait irregularity of the person, one or more cueing units, and a cueing control unit for controlling the one or more cueing units to provide, in response to the detection of the gait irregularity, a first cue set of one or more cues for treating the gait irregularity to the person. The gait irregularity monitoring unit is adapted to monitor the effect of the providing of the first cue set to the person and to determine whether the detected gait irregularity is being overcome. The cueing control unit is adapted to control the one or more cueing units to provide a different second cue set of one or more cues to the person, if it has been determined that the detected gait irregularity has not been overcome. This improves the likelihood of being able to effectively treat the detected gait irregularity.

1. A treatment apparatus for treating a gait irregularity of a person, wherein the gait irregularity is Freezing of Gait (FoG), the treatment apparatus comprising:
a gait irregularity monitoring unit for detecting a gait irregularity of the person,
one or more cueing units, and
a cueing control unit for controlling, in response to the detection of the gait irregularity, the one or more cueing units to provide a first cue set of one or more cues for treating the detected gait irregularity to the person,
wherein the gait irregularity monitoring unit is adapted to monitor the effect of the providing of the first cue set to the person and to determine whether the detected gait irregularity is being overcome, and
wherein the cueing control unit is adapted to control the one or more cueing units to provide a different second cue set of one or more cues to the person, if it has been determined that the detected gait irregularity has not been overcome.

2. The treatment apparatus as defined in claim 1, wherein the first and the second cue set differ in any one or more of:
(i) a modality of at least one cue,
(ii) that the second cue set comprises at least one cue of an additional modality compared to the first cue set or vice versa, and
(iii) that an information provided by at least one cue of a given modality differs between the first and the second cue set.

3. The treatment apparatus as defined in claim 1, wherein the cueing control unit is adapted to control the one or more cueing units to provide the first cue set to the person for a first predetermined period of time and the gait irregularity monitoring unit is adapted to determine that the detected gait irregularity has not been overcome if the detected gait irregularity has not ceased until the end of the first predetermined period of time.

4. The treatment apparatus as defined in claim 1, wherein the first and the second cue set form part of a cue set sequence of different cue sets, wherein the cueing control unit is adapted to control the one or more cueing units to successively provide the different cue sets to the person, as long as it is determined by the gait irregularity determining unit that the detected gait irregularity has not been overcome.

5. The treatment apparatus as defined in claim 4, further comprising:
a gait irregularity qualifying unit for qualifying the detected gait irregularity based on information that is indicative of any one or more of:
a current context of the person,
past gait irregularities of the person, and/
gait irregularities of a population of persons,
wherein the cueing control unit is adapted to construct the cue set sequence based on the outcome of the qualifying.

6. The treatment apparatus as defined in claim 5, wherein where the gait irregularity qualifying unit is for qualifying the detected gait irregularity based on information that is indicative of a current context of the person, the apparatus further comprises:
one or more context information providing units for providing the information that is indicative of the current context of the person.

7. The treatment apparatus as defined in claim 6, wherein the one or more context information providing units comprise:
an input unit for inputting information about a medication administration of the person,
wherein the gait irregularity qualifying unit is adapted to qualify the detected gait irregularity based on information that is indicative of a current context of the person, wherein the current context of the person comprises the current medication status of the person.

8. The treatment apparatus as defined in claim 4, wherein the gait irregularity determining unit is adapted to detect, monitor or detect and monitor the gait irregularity by means of an algorithm, and/or the cueing control unit is adapted to construct the cue set sequence by means of an algorithm,
wherein the treatment apparatus further comprises:
an update unit for allowing the algorithm(s), data that is input to the algorithm(s) or algorithm(s) and data that is input to the algorithm(s) to be updated.

9. The treatment apparatus as defined in claim 4, further comprising:
an alarm communication unit for automatically communicating an alarm to a remote location, if it is determined by the gait irregularity determining unit that, after the providing of a predetermined number of the different cue sets to the person or after a second predetermined period of time, the detected gait irregularity has still not been overcome.

10. The treatment apparatus as defined in claim 4, further comprising:
a user interface for allowing the person to input a stop command, a sensitivity change command, or a stop command and a sensitivity change command,
wherein the cueing control unit is adapted to stop, in response to the input of the stop command, the one or more cueing units to provide the different cue sets to the person, and
wherein the gait irregularity monitoring unit is adapted to change, in response to the input of the sensitivity change command, a sensitivity of the detecting of the gait irregularity of the person.

11. The treatment apparatus as defined in claim 1, further comprising:
one or more gait information sensing units for sensing information that is indicative of a current gait of the person,
wherein the gait irregularity monitoring unit is adapted to detect the gait irregularity based on the sensed information.

12. The treatment apparatus as defined in claim 11, wherein the one or more gait information sensing units comprise any one or more of: (i) an accelerometer, (ii) a gyroscope, and (iii) a muscle tension sensor.

13. A method of operating an apparatus for treating a gait irregularity of a person, wherein the gait irregularity is Freezing of Gait (FoG), the method comprising:
detecting a gait irregularity of the person, by a gait irregularity monitoring unit, and
controlling, in response to the detection of the gait irregularity, one or more cueing units to provide a first cue set of one or more cues for treating the detected gait irregularity to the person, by a cueing control unit,
wherein the effect of the providing of the first cue set to the person is monitored and it is determined whether the detected gait irregularity is being overcome, by the gait irregularity monitoring unit, and
wherein the one or more cueing units are controlled, by the cueing control unit, to provide a different second cue set of one or more cues to the person, if it has been determined that the detected gait irregularity has not been overcome.

14. A computer program product comprising a computer readable medium having computer readable program code embodied therein, the code being configured such that, on execution by a suitable processor, the processor is caused to carry out the method as defined in claim 13.
